(19) United States
(12) Patent Application Publication
(10) Pub. No.: US 2022/0031382 A1
(43) Pub. Date: Feb. 3, 2022

(54) BATTERY OPERATED TISSUE CORING DEVICE

(71) Applicant: Ethicon, Inc., Somerville, NJ (US)

(72) Inventors: William Cohn, Houston, TX (US); Matthew Kuhn, Houston, TX (US); Chris Mills, Houston, TX (US); Steven Nguyen, Houston, TX (US); Tushar Sharma, Houston, TX (US)

(21) Appl. No.: 17/368,992

(22) Filed: Jul. 7, 2021

Related U.S. Application Data
(60) Provisional application No. 63/057,523, filed on Jul. 28, 2020.

(51) Int. Cl. A61B 18/14

(52) U.S. Cl. CPC A61B 18/1445; A61B 2018/0063

(57) ABSTRACT

A surgical instrument comprises an end effector configured to core tissue, a handle operatively coupled to the end effector, wherein the handle comprises a trigger to actuate the end effector, the handle comprising a battery dock, the battery dock comprising a protruding member; and a battery unit attachable to the battery dock, wherein the battery unit is in electrical contact with at least one of the handle and the end effector when attached to the battery dock.

### CROSS-REFERENCE TO RELATED APPLICATIONS

This application claims priority to and the benefit of U.S. Patent Application No. 63/057,523 filed Jul. 28, 2020, which is hereby incorporated by reference in their entirety.

### BACKGROUND

A growing number of surgical instruments are powered by one or more battery cells. Such instruments include a variety of electrically powered implements and may be used in a variety of surgical environments.

Battery-powered surgical instruments often utilize primary cells, which are pre-charged and often intended for a single discharge (e.g., one use). Using single discharge cells may address certain difficulties associated with re-sterilizing and recharging cells. Primary cells, however, present challenges related to shipping, storage and disposal. For example, charged cells can result in hazardous waste if not properly discharged since they may be only used once and still have significant amount of charge left. To mitigate the-risks, many jurisdictions have regulations governing the conditions under which cells may be shipped and disposed. Cells and batteries with higher amounts of stored energy are required to be shipped, stored, and disposed of with safety measures that are more stringent and often more expensive.

Improvements are needed.

### SUMMARY

It may be desirable to remove a core of tissue from other target tissue sites including, but not limited to, the lungs, the liver, pancreas, or gastrointestinal (GI) tract, for which managing post-coring bleeding may be desired. A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions.

The present disclosure comprises methods and devices relating to removing a core of tissue from a tissue site. Such coring may further comprise introducing a tissue resection device to a tissue site, using the tissue resection device to create a core of tissue, removing the core of tissue from the body to create a tissue cavity, and sealing the tissue cavity.

Methods for coring tissue may comprise disposing a tissue resection device at a target tissue site, causing the tissue resection device to resect a core of tissue from the target tissue site, and removing the core of tissue from the body, wherein the removing the core of tissue from the body creates a core cavity at the target tissue site. The core of tissue comprises at least a portion of a tissue lesion. The resecting the core of tissue from the target tissue site may comprise mechanical transection. The resecting the core of tissue from the target tissue site may comprise the delivery of radiofrequency energy. The resecting the core of tissue from the target tissue site may comprise mechanical compression and the delivery of radiofrequency energy. The resecting the core of tissue from the target tissue site may comprise transection with an energized wire. The resecting the core of tissue from the target tissue site may comprise one of more of mechanical compression, the delivery of radiofrequency energy, the delivery of microwave energy, the delivery of ultrasonic energy, or transection with an energized wire. Other resection devices and procedures may be used. The resection device may be configured for one or more of mechanical compression, the delivery of radiofrequency energy, the delivery of microwave energy, the delivery of ultrasonic energy, or transection with an energized wire.

A surgical instrument comprising: an end effector comprising: a first clamping element comprising a helical coil; a second clamping element, the second clamping element being positioned to oppose at least a portion of the first clamping element; a first and second electrode configured for the delivery of radiofrequency energy for sealing tissue; and a cutting element configured for the transection of at least a portion of the sealed tissue; a handle operatively coupled to the end effector, wherein the handle comprises a trigger to actuate the end effector, the handle comprising a battery dock; the battery dock comprising a protruding member; and a battery unit attachable to the battery dock, wherein the battery unit is in electrical contact with at least one of the handle and the end effector when attached to the battery dock, and wherein the battery unit comprises: a casing; an anode and a first cathode positioned within the casing; and a translatable discharge drain, wherein, upon attachment of the battery unit to the battery dock, the protruding member contacts the discharge drain and the discharge drain translates with respect to casing to electrically couple the first anode of the battery unit to the first cathode of the battery unit.

A surgical instrument comprising: an end effector configured to core tissue; a handle operatively coupled to the end effector, wherein the handle comprises a trigger to actuate the end effector, the handle comprising a battery dock; the battery dock comprising a protruding member; and a battery unit attachable to the battery dock, wherein the battery unit is in electrical contact with at least one of the handle and the end effector when attached to the battery dock, and wherein the battery unit comprises: a casing; an anode and a first cathode positioned within the casing; and a translatable discharge drain, wherein, upon attachment of the battery unit to the battery dock, the protruding member contacts the discharge drain and the discharge drain translates with respect to casing to electrically couple the first anode of the battery unit to the first cathode of the battery unit.

A surgical instrument comprising: an end effector configured for coring tissue, the wherein the end effector comprises: a first clamping element comprising a helical coil and a first electrode; a second clamping element comprising a second electrode, the second clamping element being positioned to oppose at least a portion of the first clamping element; and a cutting element configured for the transection of tissue; and a battery assembly, comprising a control circuit comprising a battery, a memory coupled to the battery, and a processor coupled to the memory and the battery, wherein the processor is configured to generate a digital waveform; and a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform, wherein the battery assembly and the end effector are configured to mechanically and electrically connect to the handle assembly, and wherein the end effector is configured to receive the analog waveform.

A surgical instrument comprising: an end effector configured for coring tissue; a battery assembly, comprising a control circuit comprising a battery, a memory coupled to the battery, and a processor coupled to the memory and the battery, wherein the processor is configured to generate a digital waveform; and a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform, wherein the battery assembly and the end effector are configured to mechanically and electrically connect to the handle assembly, and wherein the end effector is configured to receive the analog waveform.

A surgical instrument, comprising: a control circuit comprising a memory coupled to a processor, wherein the processor is configured to cause generation of a digital waveform; a handle assembly comprising a common first stage circuit coupled to the processor, the common first stage circuit configured to receive the digital waveform, convert the digital waveform into an analog waveform, and amplify the analog waveform; and an end effector assembly configured for coring tissue, the wherein the assembly comprises: at least one clamping element comprising a helical coil, a first electrode coupled to the common first stage circuit to receive an analog waveform; element being positioned to oppose at least a portion of the clamping element; and a cutting element configured for the transection of tissue, wherein the end effector assembly is configured to mechanically and electrically connect to the handle assembly.

A surgical instrument, comprising: a control circuit comprising a memory coupled to a processor, wherein the processor is configured to cause generation of a digital waveform; a handle assembly comprising a common first stage circuit coupled to the processor, the common first stage circuit configured to receive the digital waveform, convert the digital waveform into an analog waveform, and amplify the analog waveform; and an end effector assembly configured for coring tissue, wherein the end effector assembly is configured to mechanically and electrically connect to the handle assembly.

### BRIEF DESCRIPTION OF THE DRAWINGS

The following drawings show generally, by way of example, but not by way of limitation, various examples discussed in the present disclosure. In the drawings:

FIG. 1 shows a bottom view of an example battery unit in accordance with the present disclosure.

FIG. 2 shows an isometric view of the battery unit of FIG. 1.

FIG. 3 shows a bottom view of example battery cells of the battery unit of FIG. 1 in accordance with the present disclosure.

FIG. 4 shows an isometric view of the battery cells of FIG. 2.

FIG. 5 shows an example battery dock in accordance with the present disclosure.

FIG. 6 shows a protruding member of the battery dock of FIG. 5.

FIG. 7 shows an example tissue coring device configured with a battery in accordance with the present disclosure.

FIG. 8 shows the tissue coring device of FIG. 7 with example battery cells exploded.

FIG. 9A depicts a cross-sectional side view of another exemplary power drain in an open position.

FIG. 9B depicts a cross-sectional side view the power drain of FIG. 9A in a closed position.

FIG. 10 depicts a perspective view of the power drain of FIG. 9A;

FIG. 11 depicts another exemplary battery unit attached to an exemplary dock of a handle assembly with various components omitted for clarity;

### DETAILED DESCRIPTION

The present disclosure relates to systems and methods for coring tissue. Various tissue and sites may benefit from the disclosed systems and methods.

A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions.

Certain embodiments may be directed to a surgical instrument having an end effector and a handle operatively coupled to the end effector.

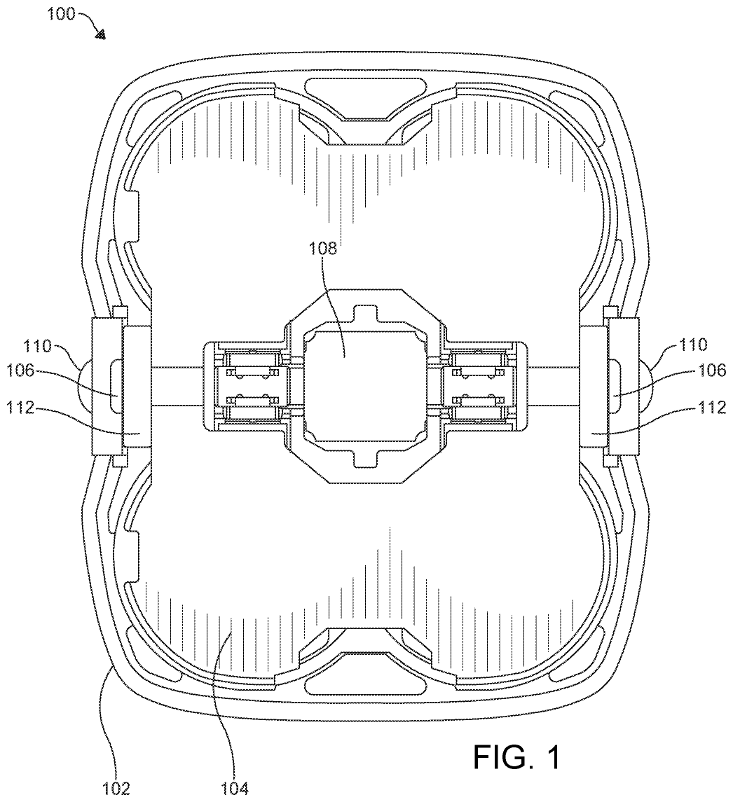

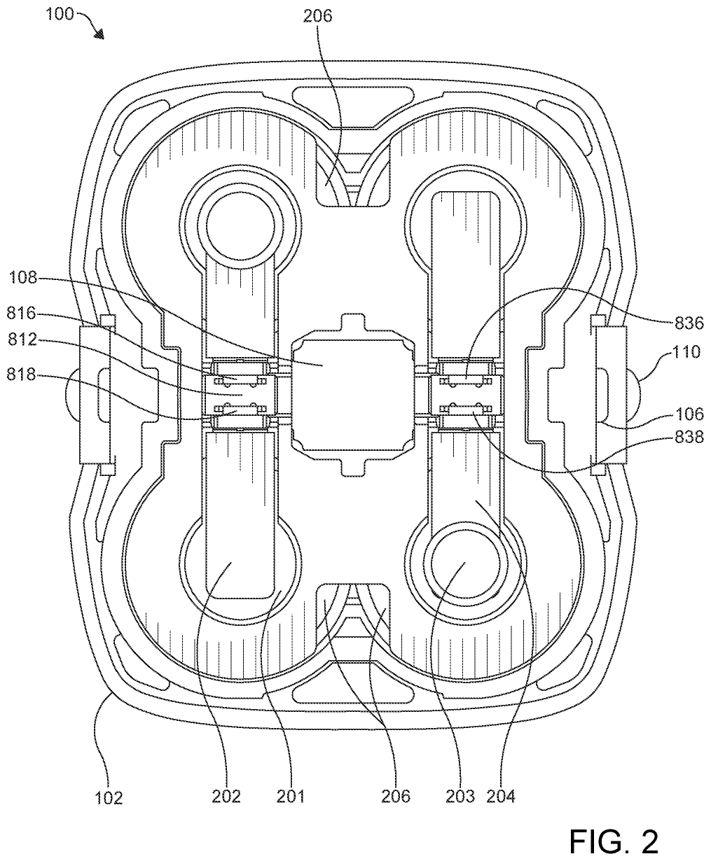

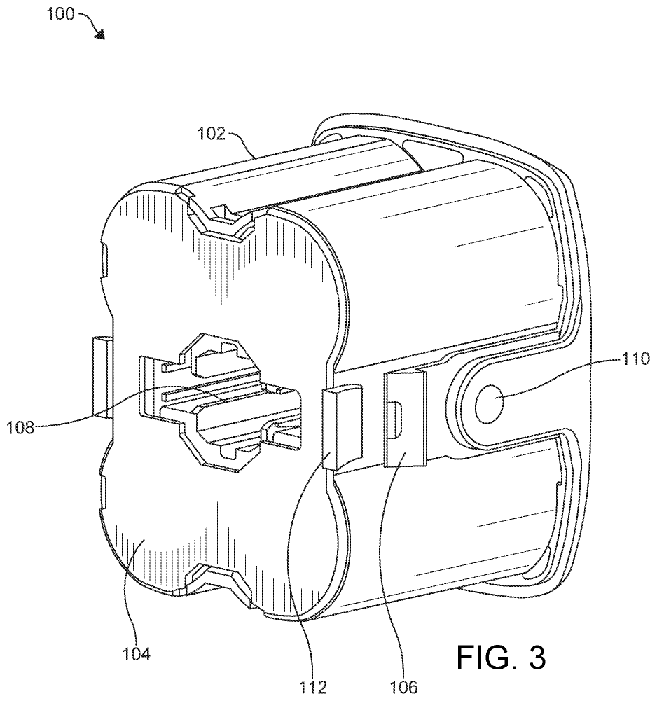

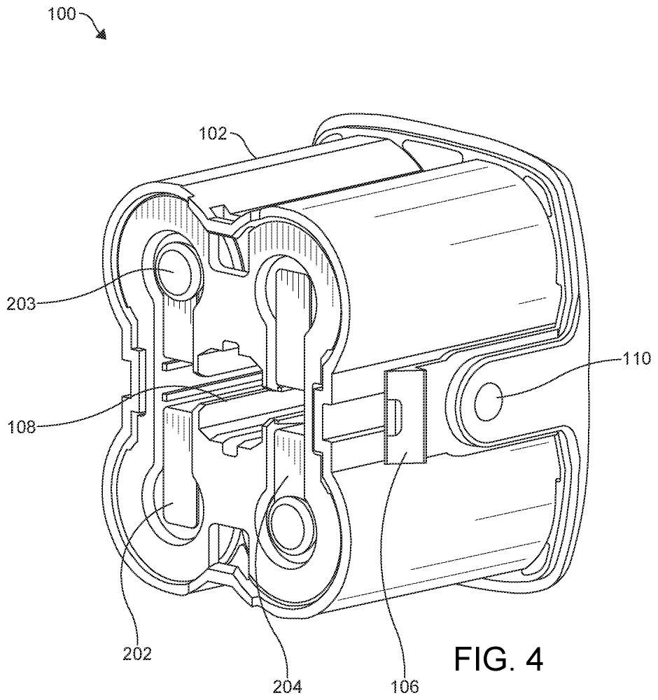

The handle may have a trigger to actuate the end effector and a battery dock that has a protruding member. As described herein, the surgical instrument may include a battery unit 100. FIG. 1 shows a bottom view of an example battery unit in accordance with the present disclosure. FIG. 2 shows an isometric view of the battery unit of FIG. 1. FIG. 3 shows a bottom view of example battery cells of the battery unit of FIG. 1 in accordance with the present disclosure. FIG. 4 shows an isometric view of the battery cells of FIG. 2.

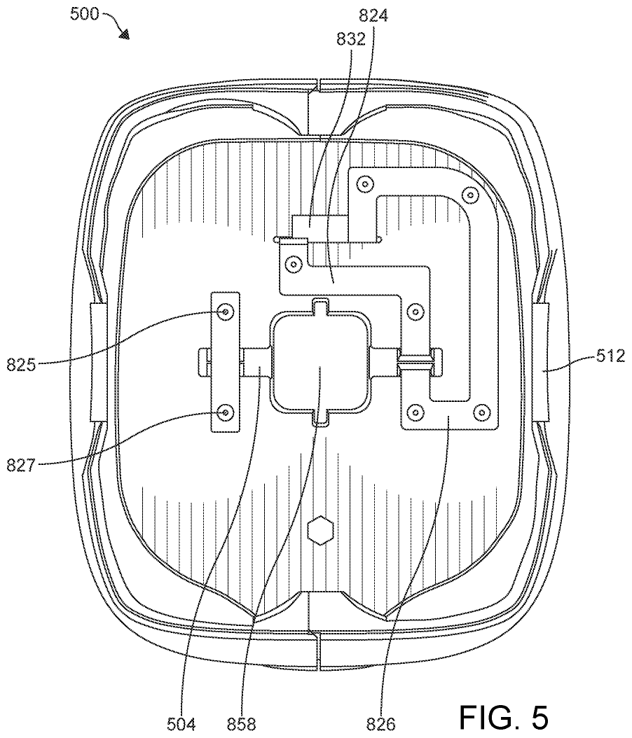

The battery unit 100 may have a casing 102 and a plurality of cells positioned within the casing, where at least a portion of the plurality of cells are not electrically connected to one another. Finger grips 110 on the sides of the casing 102 may enable the user to insert or remove the battery unit 100 from the battery dock 500 (see below). In addition, the battery unit 100 has unit clips 106 to hold it in place when inserted into the battery dock 500. The battery unit 100 has an interior cavity 108 shaped to mate with the protruding member 858 of the battery dock 500. A cover or cap 104 may cover the batteries 206 and the discharge switch from view in this embodiment. The cover 104 may be attached to the casing 102 by means of cover clips 112 as shown in this embodiment though other options such as mechanical latches may also be used. FIG. 2 depicts the battery unit 100 with the cap 104 removed to reveal the interior cavity 108 and the battery cells. The battery unit 100 may have battery cells with a cathode 201 and a cathode contact 202. The battery unit may have battery cells with an anode 203 and an anode contact 204. In the embodiment shown in FIG. 2, there are at least four cells, each with an anode and a cathode, but only one side of each cell is depicted. In the embodiment shown in FIG. 2, the bottom of the battery unit 100 shows four cells with two cathodes 201 exposed and two anodes exposed 203, along with two cathode contacts 202 and two anode contacts 204. For simplicity a single set of each is labelled. The battery unit 100 may have a discharge switch with contacts 816, 818, 836, 838. The contacts may contact electrodes (see FIG. 5 below) when the discharge switch is in the closed position. When in the open position the discharge switch contacts are not in electrical contact with the battery cells. When in the closed position, the contacts 816, 818, 836, 838 contact their respective electrodes (see FIG. 5 below, items 824, 825, 826, 827) and slowly drain the batteries of the unit by connecting the anode with the cathode through a circuit 832 with at least one resistive element (not shown). Batteries or cells 206 reside within the battery unit 100.

The discharge switch may be mechanically biased towards a position, where the discharge switch is held in the open position by a non-conductive portion of the casing. The discharge switch may be translated into the closed position by the protruding member 858 upon attachment of the battery unit 100 into the battery compartment of a surgical instrument.

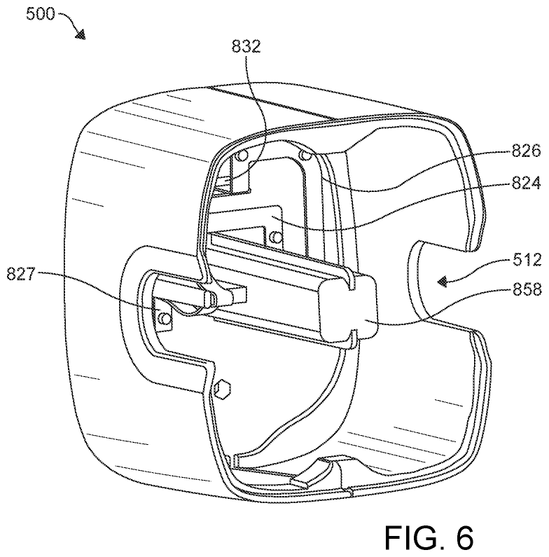

Battery unit 100 may be attachable to a battery dock 500 in this embodiment by means of unit clips 106. FIG. 5 shows an example battery dock 500 in accordance with the present disclosure. The battery dock 500 may have side regions 512 for matching with the battery unit clips 106 and finger grips 110. The battery dock 500 may have electrodes 824, 825, 826, 827 connecting to a circuit 832 comprising at least a resistive element which may slowly discharge the batteries when the contacts 816, 818, 836, 838 are in contact with the electrodes. FIG. 6 is an isometric view of the battery dock 500. FIG. 6 shows interior features of the battery dock of FIG. 5, including a protruding member 858. The battery unit 100 may be in electrical contact with at least one of the handle and the end effector when attached to the battery dock 500. The battery unit 100 may have a casing 102 and a first anode 201 and a first cathode 203 positioned within the casing 102. The battery unit 100 may also have a translatable discharge drain 812, where, upon attachment of the battery unit 100 to the battery dock 500, the protruding member 858 contacts the discharge drain 812 and the discharge drain 812 translates with respect to the casing 102 to electrically couple the batteries to the circuit 832 with at least one resistive element so that the batteries may slowly drain of charge.

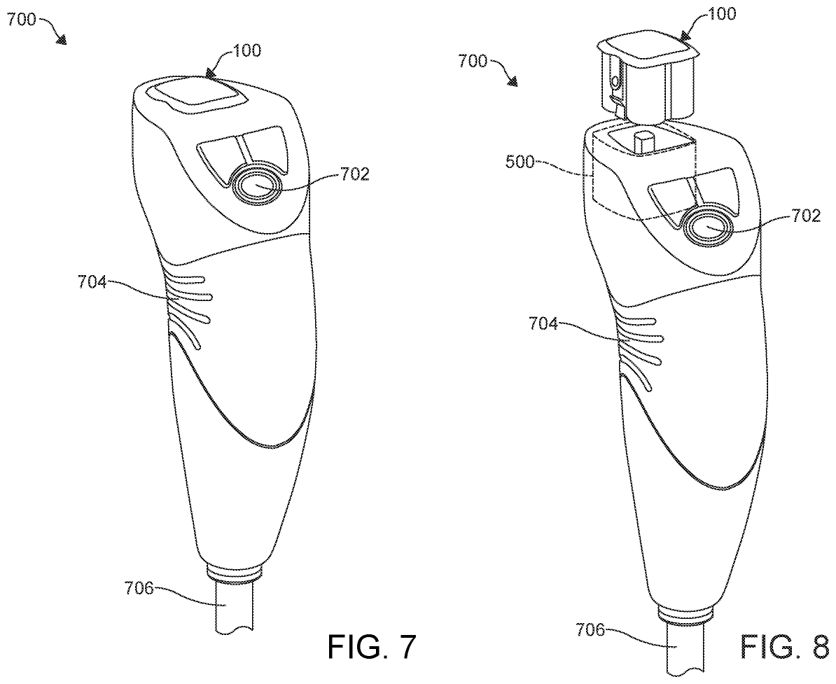

Also, various embodiments may be directed to a surgical instrument comprising a battery dock 500 and a battery unit 100. FIG. 7 shows an example surgical device (e.g., a tissue coring device) configured with a battery in accordance with the present disclosure. The example surgical devices comprises a hand grip 704 and instrument controls 702 and a connection 706 to the end effector. FIG. 7 depicts the battery unit 100 in place in the surgical device. FIG. 8 shows the tissue coring device of FIG. 7 with an example battery unit 100 and battery dock 500 exploded. The surgical instrument may include a battery dock 500 further comprising a protruding member (e.g., the protruding member 858 of FIG. 6) positioned proximate the battery unit 100.

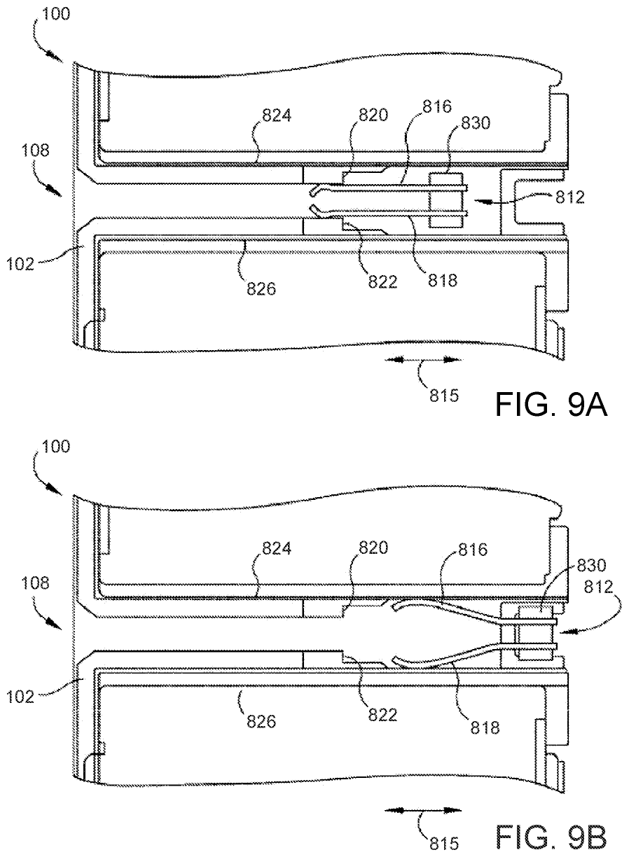

FIGS. 9A and 9B illustrate cross-sectional views of an exemplary battery unit 100 including a translatable power drain 812. Power drain 812 may be positioned within interior cavity 108 and may be translatable within interior cavity 108 in the directions of arrow 815. FIG. 9A shows power drain 812 in an open position and FIG. 9B shows power drain 812 in a closed position. The power drain 812 may comprise at least two contacts 816, 818. When the power drain 812 is in the open position, a portion of contacts 816, 818 may touch a non-conductive portion of casing 102, such as fingers 820, 822. In the present example, contacts 816, 818 are resiliently biased to exert a force against fingers 820, 822 in order to resist movement of the power drain 812 in the direction of arrows 815. Also, fingers 820, 822 may define one or more protrusions or stepped down portions, as shown in FIGS. 9A and 9B. Battery unit 100 of the present example also comprises electrodes 824, 826. Electrodes 824, 826 may each be electrically coupled to a cathode or an anode of cells contained within battery unit 100. In the closed position (FIG. 9B), contacts 816, 818 may be in electrical connection with electrodes 824, 826, thereby allowing the voltage source to discharge through power drain 812. As discussed in more detail elsewhere, power drain 812 may be transitioned from the open position to the closed position upon attachment of battery unit 100 to a battery dock 500.

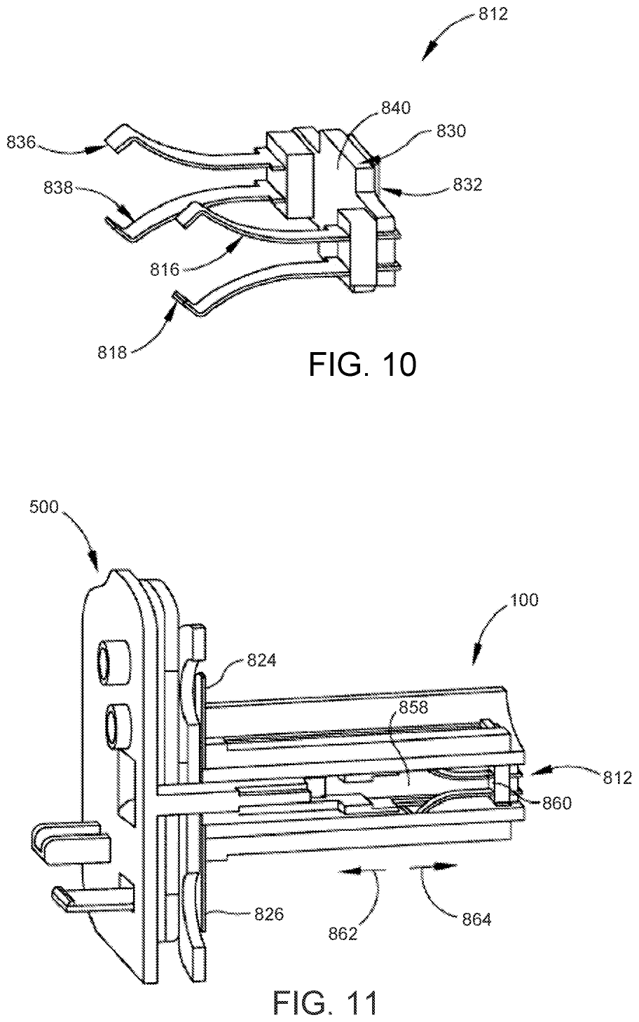

FIG. 10 is a perspective view of power drain 812 in accordance with one non-limiting example. Contacts 816, 818 of drain 812 may be coupled to a base portion 830 of drain 812. Similarly, a second pair of contacts 836, 838 of drain 812 may be coupled to base portion 830 of drain 812. According to various examples, contacts 816, 818 may be electrically connected to one another via a resistive element (not shown) as part of a circuit 832. Similarly, contacts 836, 838 may be electrically connected to one another via a circuit 832 which includes a resistive element. As illustrated, contacts 816, 818, 836, 838 may have a bend or curvature to resiliently bias contacts 816, 818, 836, 838 toward an outward position when inwardly compressed. Additionally, in the present example, distal end of each of contacts 816, 818, 836, 838 has an inwardly turned section. Base portion 830 comprises a contacting surface 840 that engages surgical instrument 500 when battery unit 100 is attached to surgical instrument 500. Through this engagement, battery drain 812 may be translated relative to casing 100.

FIG. 11 illustrates battery unit 100 attached to a battery dock 500. For clarity, various components have been removed. Referring now to FIGS. 9A-11, battery dock 500 comprises a protruding member 858 that is sized to be received by cavity 108 (see FIG. 3 and FIG. 4) of battery unit 100. Prior to attachment, power drain 812 is in the open position (FIG. 9A). During attachment of battery unit 100 to battery dock 500, protruding member 858 is inserted into the cavity 108 and battery unit 100 is moved relative to battery dock 500 in the direction indicated by arrow 862. Eventually distal end 860 of protruding member 858 contacts contacting surface 840 of power drain 812. As the operator continues to attach battery unit 100, power drain 812 translates relative to casing 102 in the direction indicated by arrow 864 and moves to the closed position (see FIG. 12B). In this closed position, battery unit 100 commences to slowly drain. When the battery unit 100 is removed from battery dock 500, power drain 812 may remain in the position shown in FIG. 12B. In this way, the cells (not shown) of battery unit 100 may drain any remaining charge across a resistive element either before or during disposal.

Additionally, various embodiments may be directed to a surgical system having a surgical device having a battery dock. The surgical system may also have a battery unit 100, where the battery unit has a first and second grouping of cells and a translatable battery drain 812 positioned proximate the first and second grouping of cells. The translatable battery drain 812 may have a first and second set of contacts (816, 818, 836, 838); where, in a first position, the first and second set of contacts are not electrically coupled to first and second grouping of cells. In a second position, the first set of contacts may be electrically coupled to the first grouping of cells and the second set of contacts is electrically coupled to the second grouping of cells. The translatable battery drain 812 may translate from the first position to the second position upon attachment of the battery unit 100 to the battery dock 500.

A replaceable battery confers several advantages to the use of the tissue coring device. By not having an attached power cord, the user is free to rotate the device (and similarly the helical coil end effector) into the target tissue site without worrying about the power cord wrapping around the device. A battery operated device also may not require a complex system of gears and motors to automatically rotate the device to gradually cut and set a core of tissue. Instead, the rotation may be manually applied by the user. This helps simplify the design of the internal components of the tissue coring device.

Electrosurgical instruments for applying electrical energy to tissue in order to treat and/or destroy the tissue are finding increasingly widespread applications in surgical procedures. Depending upon specific instrument configurations and operational parameters, electrosurgical instruments can provide simultaneous or near-simultaneous cutting of tissue and hemostasis by coagulation, desirably minimizing patient trauma. An electrosurgical instrument typically includes a hand piece, an instrument having a distally-mounted end effector (e.g., one or more electrodes). The end effector can be positioned against the tissue such that electrical current is introduced into the tissue. Electrosurgical instruments can be configured for bipolar or monopolar operation. During bipolar operation, current is introduced into and returned from the tissue by active and return electrodes, respectively, of the end effector. During monopolar operation, current is introduced into the tissue by an active electrode of the end effector and returned through a return electrode (e.g., a grounding pad) separately located on a patient's body. Heat generated by the current flowing through the tissue may form hemostatic seals within the tissue and/or between tissues and thus may be particularly useful for sealing blood vessels, for example. The end effector of an electrosurgical instrument also may include a cutting member that is movable relative to the tissue and the electrodes to transect the tissue.

Electrical energy applied by an electrosurgical instrument can be transmitted to the instrument by a generator in communication with the hand piece. The electrical energy may be in the form of radio frequency (“RF”) energy. RF energy is a form of electrical energy that may be in the frequency range of 200 kilohertz (kHz) to 1 megahertz (MHz). In application, an electrosurgical instrument can transmit low frequency RF energy through tissue, which causes ionic agitation, or friction, in effect resistive heating, thereby increasing the temperature of the tissue. Because a sharp boundary is created between the affected tissue and the surrounding tissue, surgeons can operate with a high level of precision and control, without sacrificing un-targeted adjacent tissue. The low operating temperatures of RF energy is useful for removing, shrinking, or sculpting soft tissue while simultaneously sealing blood vessels. RF energy works particularly well on connective tissue, which is primarily comprised of collagen and shrinks when contacted by heat.

The RF energy may be in a frequency range described in EN 60601-2-2:2009+A11:2011, Definition 201.3.218—HIGH FREQUENCY. For example, the frequency in monopolar RF applications may be typically restricted to less than 5 MHz. However, in bipolar RF applications, the frequency can be almost anything. Frequencies above 200 kHz can be typically used for monopolar applications in order to avoid the unwanted stimulation of nerves and muscles that would result from the use of low frequency current. Lower frequencies may be used for bipolar applications if the risk analysis shows the possibility of neuromuscular stimulation has been mitigated to an acceptable level. Normally, frequencies above 5 MHz are not used in order to minimize the problems associated with high frequency leakage currents. Higher frequencies may, however, be used in the case of bipolar applications. It is generally recognized that 10 mA is the lower threshold of thermal effects on tissue.

In one aspect, the present disclosure provides a surgical instrument. The surgical instrument comprises a battery assembly, comprising a control circuit comprising a battery, a memory coupled to the battery, and a processor coupled to the memory and the battery, wherein the processor is configured to generate a digital waveform; a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform; and an end effector assembly comprising a clamping element in the form of a helical coil shaped electrode capable of receiving the analog waveform, and apply the analog waveform to a load; wherein the battery assembly and the end effector assembly are configured to mechanically and electrically connect to the handle assembly.

In another aspect, the present disclosure provides a surgical instrument. The surgical instrument comprises a control circuit comprising a memory coupled to a processor, wherein the processor is configured to generate a digital waveform; a handle assembly comprising a common first stage circuit coupled to the processor, the common first stage circuit configured to receive the digital waveform, convert the digital waveform into an analog waveform, and amplify the analog waveform; and an end effector assembly comprising a helical electrode coupled to the common first stage amplifier circuit to receive the analog waveform; wherein the end effector assembly is configured to mechanically and electrically connect to the handle assembly.

In one or more various aspects, related systems include but are not limited to circuitry and/or programming for effecting herein-referenced method aspects; the circuitry and/or programming can be virtually any combination of hardware, software, and/or firmware configured to affect the herein-referenced method aspects depending upon the design choices of the system designer. Further, it is understood that any one or more of the described forms, expressions of forms, examples, can be combined with any one or more of the other following-described forms, expressions of forms, and examples.

The present disclosure comprises at least the following aspects:

Aspect 1. A surgical instrument comprising: an end effector comprising: a first clamping element comprising a helical coil; a second clamping element, the second clamping element being positioned to oppose at least a portion of the first clamping element; a first and second electrode configured for delivery of radiofrequency energy for sealing tissue; and a cutting element configured for transection of at least a portion of the sealed tissue; a handle operatively coupled to the end effector, wherein the handle comprises a trigger to actuate the end effector, the handle comprising a battery dock; the battery dock comprising a protruding member; and a battery unit attachable to the battery dock, wherein the battery unit is in electrical contact with at least one of the handle and the end effector when attached to the battery dock, and wherein the battery unit comprises: a casing; an anode and a first cathode positioned within the casing; and a translatable discharge drain, wherein, upon attachment of the battery unit to the battery dock, the protruding member contacts the discharge drain and the discharge drain translates with respect to casing to electrically couple the first anode of the battery unit to the first cathode of the battery unit.

Aspect 2. The surgical instrument of aspect 1, wherein the discharge drain comprises a first contact and a second contact, and at least one resistive element electrically coupled to the first and second contact, wherein, upon attachment of the battery unit to the battery dock, the discharge drain electrically couples the anode to the cathode through the at least one resistive element.

Aspect 3. The surgical instrument of aspect 2, wherein the at least one resistive element has a resistance in a range of about 90 ohms to about 110 ohms.

Aspect 4. The surgical instrument of any one of aspects 1-3, wherein the anode comprises a first anode and the cathode comprises a first cathode, wherein the battery unit comprises a second anode and a second cathode positioned within the casing and wherein, upon attachment of the battery unit to the battery dock, the discharge drain electrically couples the second anode to the second cathode.

Aspect 5. The surgical instrument of any one of aspects 1-4, wherein the battery unit comprises at least one cell selected from the group consisting of a CR123 cell and a CR2 cell.

Aspect 6. A surgical instrument comprising: an end effector configured to core tissue; a handle operatively coupled to the end effector, wherein the handle comprises a trigger to actuate the end effector, the handle comprising a battery dock; the battery dock comprising a protruding member; and a battery unit attachable to the battery dock, wherein the battery unit is in electrical contact with at least one of the handle and the end effector when attached to the battery dock, and wherein the battery unit comprises: a casing; an anode and a first cathode positioned within the casing; and a translatable discharge drain, wherein, upon attachment of the battery unit to the battery dock, the protruding member contacts the discharge drain and the discharge drain translates with respect to casing to electrically couple the first anode of the battery unit to the first cathode of the battery unit.

Aspect 7. The surgical instrument of aspect 6, wherein the discharge drain comprises a first contact and a second contact, and at least one resistive element electrically coupled to the first and second contact, wherein, upon attachment of the battery unit to the battery dock, the discharge drain electrically couples the anode to the cathode through the at least one resistive element.

Aspect 8. The surgical instrument of aspect 7, wherein the at least one resistive element has a resistance in a range of about 90 ohms to about 110 ohms.

Aspect 9. The surgical instrument of any one of aspects 6-8, wherein the anode comprises a first anode and the cathode comprises a first cathode, wherein the battery unit comprises a second anode and a second cathode positioned within the casing and wherein, upon attachment of the battery unit to the battery dock, the discharge drain electrically couples the second anode to the second cathode.

Aspect 10. The surgical instrument of any one of aspects 6-9, wherein the battery unit comprises at least one cell selected from the group consisting of a CR123 cell and a CR2 cell.

Aspect 11. A surgical instrument comprising: an end effector configured for coring tissue, wherein the end effector comprises: a first clamping element comprising a helical coil and a first electrode; a second clamping element comprising a second electrode, the second clamping element being positioned to oppose at least a portion of the first clamping element; and a cutting element configured for transection of tissue; and a battery assembly, comprising a control circuit comprising a battery, a memory coupled to the battery, and a processor coupled to the memory and the battery, wherein the processor is configured to generate a digital waveform; and a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform, wherein the battery assembly and the end effector are configured to mechanically and electrically connect to the handle assembly, and wherein the end effector is configured to receive the analog waveform.

Aspect 12. A surgical instrument comprising: an end effector configured for coring tissue; a battery assembly, comprising a control circuit comprising a battery, a memory coupled to the battery, and a processor coupled to the memory and the battery, wherein the processor is configured to generate a digital waveform; and a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform, wherein the battery assembly and the end effector are configured to mechanically and electrically connect to the handle assembly, and wherein the end effector is configured to receive the analog waveform.

Aspect 13. A surgical instrument, comprising: a control circuit comprising a memory coupled to a processor, wherein the processor is configured to cause generation of a digital waveform; a handle assembly comprising a common first stage circuit coupled to the processor, the common first stage circuit configured to receive the digital waveform, convert the digital waveform into an analog waveform, and amplify the analog waveform; and an end effector assembly configured for coring tissue, wherein the assembly comprises: at least one clamping element comprising a helical coil, a first electrode coupled to the common first stage circuit to receive an analog waveform; element being positioned to oppose at least a portion of the at least one clamping element; and a cutting element configured for transection of tissue, wherein the end effector assembly is configured to mechanically and electrically connect to the handle assembly.

Aspect 14. A surgical instrument, comprising: a control circuit comprising a memory coupled to a processor, wherein the processor is configured to cause generation of a digital waveform; a handle assembly comprising a common first stage circuit coupled to the processor, the common first stage circuit configured to receive the digital waveform, convert the digital waveform into an analog waveform, and amplify the analog waveform; and an end effector assembly configured for coring tissue, wherein the end effector assembly is configured to mechanically and electrically connect to the handle assembly.

Aspect 15. A surgical instrument comprising: a handle operatively coupled to an instrument, wherein the handle comprises a trigger to actuate a function of the instrument, the handle comprising a battery dock; the battery dock comprising a protruding member; and a battery unit attachable to the battery dock, wherein the battery unit is in electrical contact with at least one of the handle and the instrument when attached to the battery dock, and wherein the battery unit comprises: a casing; an anode and a first cathode positioned within the casing; and a translatable discharge drain, wherein, upon attachment of the battery unit to the battery dock, the protruding member contacts the discharge drain and the discharge drain translates with respect to casing to electrically couple the first anode of the battery unit to the first cathode of the battery unit.

Aspect 16. The surgical instrument of aspect 15, wherein the discharge drain comprises a first contact and a second contact, and at least one resistive element electrically coupled to the first and second contact, wherein, upon attachment of the battery unit to the battery dock, the discharge drain electrically couples the anode to the cathode through the at least one resistive element.

Aspect 17. The surgical instrument of aspect 16, wherein the at least one resistive element has a resistance in a range of about 90 ohms to about 110 ohms.

Aspect 18. The surgical instrument of any one of aspects 15-17, wherein the anode comprises a first anode and the cathode comprises a first cathode, wherein the battery unit comprises a second anode and a second cathode positioned within the casing and wherein, upon attachment of the battery unit to the battery dock, the discharge drain electrically couples the second anode to the second cathode.

Aspect 19. The surgical instrument of any one of aspects 15-17, wherein the battery unit comprises at least one cell selected from the group consisting of a CR123 cell and a CR2 cell.

Although shown and described is what is believed to be the most practical and preferred embodiments, it is apparent that departures from specific designs and methods described and shown will suggest themselves to those skilled in the art and may be used without departing from the spirit and scope of the invention. For example, the systems, devices and methods described herein for removal of lesions from the lung. It will be appreciated by the skilled artisan that the devices and methods described herein may are not limited to the lung and could be used for tissue resection and lesion removal in other areas of the body. The present invention is not restricted to the particular constructions described and illustrated, but should be constructed to cohere with all modifications that may fall within the scope of the appended claims.

What is claimed is:

1. A surgical instrument comprising:
an end effector comprising:
a first clamping element comprising a helical coil;
a second clamping element, the second clamping element being positioned to oppose at least a portion of the first clamping element;
a first and second electrode configured for delivery of radiofrequency energy for sealing tissue; and
a cutting element configured for transection of at least a portion of the sealed tissue;
a handle operatively coupled to the end effector, wherein the handle comprises a trigger to actuate the end effector, the handle comprising a battery dock;
the battery dock comprising a protruding member; and
a battery unit attachable to the battery dock, wherein the battery unit is in electrical contact with at least one of the handle and the end effector when attached to the battery dock, and wherein the battery unit comprises:
a casing;
an anode and a cathode positioned within the casing; and
a translatable discharge drain, wherein, upon attachment of the battery unit to the battery dock, the protruding member contacts the discharge drain and the discharge drain translates with respect to casing to electrically couple the anode of the battery unit to the cathode of the battery unit.

2. The surgical instrument of claim 1, wherein the discharge drain comprises a first contact and a second contact, and at least one resistive element electrically coupled to the first and second contact, wherein, upon attachment of the battery unit to the battery dock, the discharge drain electrically couples the anode to the cathode through the at least one resistive element.

3. The surgical instrument of claim 1, wherein the anode comprises a first anode and the cathode comprises a first cathode, wherein the battery unit comprises a second anode and a second cathode positioned within the casing and wherein, upon attachment of the battery unit to the battery dock, the discharge drain electrically couples the second anode to the second cathode.

4. A surgical instrument comprising:
an end effector configured to core tissue;
a handle operatively coupled to the end effector, wherein the handle comprises a trigger to actuate the end effector, the handle comprising a battery dock;
the battery dock comprising a protruding member; and
a battery unit attachable to the battery dock, wherein the battery unit is in electrical contact with at least one of the handle and the end effector when attached to the battery dock, and wherein the battery unit comprises:
a casing;
an anode and a cathode positioned within the casing; and
a translatable discharge drain, wherein, upon attachment of the battery unit to the battery dock, the protruding member contacts the discharge drain and the discharge drain translates with respect to casing to electrically couple the anode of the battery unit to the cathode of the battery unit.

5. The surgical instrument of claim 4, wherein the discharge drain comprises a first contact and a second contact, and at least one resistive element electrically coupled to the first and second contact, wherein, upon attachment of the battery unit to the battery dock, the discharge drain electrically couples the anode to the cathode through the at least one resistive element.

6. The surgical instrument of claim 4, wherein the anode comprises a first anode and the cathode comprises a first cathode, wherein the battery unit comprises a second anode and a second cathode positioned within the casing and wherein, upon attachment of the battery unit to the battery dock, the discharge drain electrically couples the second anode to the second cathode.

7. A surgical instrument comprising:
an end effector configured for coring tissue, wherein the end effector comprises:
a first clamping element comprising a helical coil and a first electrode;
a second clamping element comprising a second electrode, the second clamping element being positioned to oppose at least a portion of the first clamping element; and
a cutting element configured for transection of tissue; and
a battery assembly, comprising a control circuit comprising a battery, a memory coupled to the battery, and a processor coupled to the memory and the battery, wherein the processor is configured to generate a digital waveform; and
a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform,
wherein the battery assembly and the end effector are configured to mechanically and electrically connect to the handle assembly, and
wherein the end effector is configured to receive the analog waveform.

8. A surgical instrument comprising:
an end effector configured for coring tissue;
a battery assembly, comprising a control circuit comprising a battery, a memory coupled to the battery, and a processor coupled to the memory and the battery, wherein the processor is configured to generate a digital waveform; and
a handle assembly comprising a first stage circuit coupled to the processor, the first stage circuit comprising a digital-to-analog (DAC) converter and a first stage amplifier circuit, wherein the DAC is configured to receive the digital waveform and convert the digital waveform into an analog waveform, wherein the first stage amplifier circuit is configured to receive and amplify the analog waveform,
wherein the battery assembly and the end effector are configured to mechanically and electrically connect to the handle assembly, and
wherein the end effector is configured to receive the analog waveform.

9. A surgical instrument, comprising:
a control circuit comprising a memory coupled to a processor, wherein the processor is configured to cause generation of a digital waveform;
a handle assembly comprising a common first stage circuit coupled to the processor, the common first stage circuit configured to receive the digital waveform, convert the digital waveform into an analog waveform, and amplify the analog waveform; and
an end effector assembly configured for coring tissue, wherein the end effector assembly comprises:
at least one clamping element comprising a helical coil, a first electrode coupled to the common first stage circuit to receive an analog waveform;
element being positioned to oppose at least a portion of the at least one clamping element; and
a cutting element configured for transection of tissue,
wherein the end effector assembly is configured to mechanically and electrically connect to the handle assembly.

10. A surgical instrument, comprising:
a control circuit comprising a memory coupled to a processor, wherein the processor is configured to cause generation of a digital waveform;
a handle assembly comprising a common first stage circuit coupled to the processor, the common first stage circuit configured to receive the digital waveform, convert the digital waveform into an analog waveform, and amplify the analog waveform; and
an end effector assembly configured for coring tissue, wherein the end effector assembly is configured to mechanically and electrically connect to the handle assembly.

11. A surgical instrument comprising:
a handle operatively coupled to an instrument, wherein the handle comprises a trigger to actuate a function of the instrument, the handle comprising a battery dock;
the battery dock comprising a protruding member; and
a battery unit attachable to the battery dock, wherein the battery unit is in electrical contact with at least one of the handle and the instrument when attached to the battery dock, and wherein the battery unit comprises:
a casing;
an anode and a first cathode positioned within the casing; and
a translatable discharge drain, wherein, upon attachment of the battery unit to the battery dock, the protruding member contacts the discharge drain and the discharge drain translates with respect to casing to electrically couple the first anode of the battery unit to the first cathode of the battery unit.

12. The surgical instrument of claim 11, wherein the discharge drain comprises a first contact and a second contact, and at least one resistive element electrically coupled to the first and second contact, wherein, upon attachment of the battery unit to the battery dock, the discharge drain electrically couples the anode to the cathode through the at least one resistive element.

13. The surgical instrument of claim 11, wherein the anode comprises a first anode and the cathode comprises a first cathode, wherein the battery unit comprises a second anode and a second cathode positioned within the casing and wherein, upon attachment of the battery unit to the battery dock, the discharge drain electrically couples the second anode to the second cathode.
